(19) United States
(12) Patent Application Publication
(10) Pub. No.: US 2015/0223902 A1
(43) Pub. Date: Aug. 13, 2015

(54) NAVIGATION WITH 3D LOCALIZATION USING 2D IMAGES

(71) Applicant: Hansen Medical, Inc., Mountain View, CA (US)

(72) Inventors: Sean P. Walker, Fremont, CA (US); June Park, San Jose, CA (US)

(21) Appl. No.: 14/605,280

(22) Filed: Jan. 26, 2015

Related U.S. Application Data
(60) Provisional application No. 61/937,203, filed on Feb. 7, 2014.

(51) Int. Cl. A61B 19/00; A61F 2/95; A61F 2/88; A61B 5/05; A61B 5/00; A61B 17/12; A61M 25/09

(52) U.S. Cl. CPC A61B 19/5244; A61B 17/12013; A61F 2/95; A61M 25/09; A61B 5/05; A61B 5/6852; A61B 17/12118; A61B 19/56; A61F 2/88; A61B 2019/5238; A61B 2019/5265; A61B 2019/5291; A61B 2017/00292

(57) ABSTRACT

A method for facilitating a medical or surgical procedure in an operating site in a body of a patient may involve: displaying a first point on a first two-dimensional image of the operating site into which an elongate, flexible catheter device is inserted in response to a first user input; mapping the first point on at least a second two-dimensional image of the operating site, the second two-dimensional image being oriented at a non-zero angle with respect to the first two-dimensional image; displaying a first line on the second image that projects from the first point; displaying a second point on the second image in response to a second user input; and determining a three-dimensional location within the operating site, based on the first line and the second point on the second image.

### CROSS-REFERENCES TO RELATED APPLICATIONS

This application claims priority to U.S. Provisional Application No. 61/937,203, filed on Feb. 7, 2014 and entitled “NAVIGATION WITH 3D LOCALIZATION USING 2D IMAGES,” the content of which is incorporated by reference herein in its entirety.

### BACKGROUND

Navigating a catheter in a three-dimensional environment using only a two-dimensional fluoroscopy imaging projection is a challenging task, primarily because two-dimensional images, by definition, cannot show depth associated with the image shown. Accordingly, two-dimensional images may not properly convey a true position or orientation of objects. In the context of catheter systems, this can lead to errors, for example, where it is difficult or impossible to perceive exactly how a catheter or component thereof is bent or oriented within an operating site of a patient.

While some catheter systems include three-dimensional localization, a number of catheter systems do not support three-dimensional localization. Additionally, three-dimensional localization may not be practical for all applications. Accordingly, there is a need for an improved system and method for navigating a catheter where three-dimensional localization is unavailable.

### BRIEF SUMMARY

In one aspect, a method for facilitating a medical or surgical procedure in an operating site in a body of a patient may involve: displaying a first point on a first two-dimensional image of the operating site into which an elongate, flexible catheter device is inserted in response to a first user input; mapping the first point on at least a second two-dimensional image of the operating site, the second two-dimensional image being oriented at a non-zero angle with respect to the first two-dimensional image; displaying a first line on the second image that projects from the first point; displaying a second point on the second image in response to a second user input; and determining a three-dimensional location within the operating site, based on the first line and the second point on the second image.

In some embodiments, the first two-dimensional image may be an image generated with a fluoroscopic imaging system. In various embodiments, the catheter device may be any suitable device, such as but not limited to a procedural catheter, an endograft delivery catheter, a catheter sheath or a catheter guidewire. In some embodiments, the catheter device may include an electromagnetic sensor or other sensor. Optionally, the method may further include displaying a connecting line connecting the first and second points on the second image in response to an additional user input. The method may also further include displaying a number label for one or more of the points on the first image and/or the second image. In some embodiments, the method may involve displaying all the points and all the number labels on both the first image and also the second image.

In some embodiments, the first and second points may have different colors. In some embodiments, the method may further involve displaying the first and second images on either one or two display monitors. In some embodiments, the first image is derived from an imaging modality, and the method further involves generating the second image from the first image and displaying the second image. In some embodiments, generating and displaying the second image may involve generating and displaying a representation of at least part of the catheter device and at least part of the operating site. In some embodiments, generating and displaying the second image may further involve generating and displaying a representation of a background of the operating site.

In some embodiments, the operating site may be an abdominal aortic aneurysm, and the catheter may be an endograft delivery catheter. In some embodiments, the first image may be an image acquired from an imaging system oriented in a first orientation relative to the patient, such as anterior-posterior or posterior-anterior, and the second image may be an image oriented in a second orientation relative to the patient, such as left lateral and right lateral. In some embodiments, displaying the points comprises displaying points at an ostium of a blood vessel. In some embodiments, the method may include displaying an ellipse that connects the first point and the second point on the first image and/or the second image. In some embodiments, determining the three-dimensional location may involve using a least squares method of mathematical calculation.

In another aspect, a system, for facilitating a medical or surgical procedure in an operating site in a body of a patient may include: an elongate, flexible catheter device and

a visualization system. The visualization system may include a user interface, an image generator and a processor. The user interface is for accepting user inputs related to locations of items on at least two displayed images. The image generator is configured to generate images of at least a first point on a first image of the operating site and a second point on a second image of the operating site in response to the user inputs, where the first image is acquired via an imaging modality, and where the second image has an orientation that is different from an orientation of the first image. The processor is configured to map the first point on the first image with an equivalent first point on the second image, generate a first line for display on the second image that projects from the first point, and determine a three-dimensional location within the operating site, based on the first line and the second point on the second image.

In various embodiments, the catheter device may be a procedural catheter, an endograft delivery catheter, a catheter sheath, a catheter guidewire or the like. In some embodiments, the catheter device may include an electromagnetic sensor or other sensor. In some embodiments, the visualization system may further include at least one video display monitor. For example, the visualization system may include a first video display monitor for displaying the first image and a second video display monitor for displaying the second image.

In some embodiments, the imaging modality may be fluoroscopy, and the visualization system may include a connection for connecting to a fluoroscopic imaging system. In some embodiments, the processor of the visualization system is further configured to generate at least a second line connecting multiple points on at least the second image in response to the user inputs. In some embodiments, the user interface may be a touchscreen, a keyboard and/or a mouse.

In another aspect, a method for a procedure in a blood vessel of a patient may involve: advancing an elongate, flexible catheter device to an operating site in the blood vessel; viewing a first two-dimensional image of the catheter and the operating site on a display monitor; selecting a first location for a first point on the first image; viewing a second two-dimensional image of the catheter and the operational site, wherein second image includes a first line projecting from the first point; selecting a second location for a second point on the second image; and manipulating the catheter in the operating site, based at least in part on the viewing of the first and second images and the locations of the first and second points on the images.

In some embodiment, the first two-dimensional image may be an image generated with a fluoroscopic imaging system. In some embodiments, the method may further involve electing a third location for a third point on the second image and selecting at least two of the points to be connected by a connecting line. The method may also optionally involve selecting whether the connecting line is straight or curved. In some embodiments, selecting the points may involve drawing the connecting line between the points. Some embodiments may also include selecting at least a fourth location for a fourth point on the second image, where the connecting line connects at least three of the points on the second image. The method may also optionally include selecting multiple subsets of the selected points to be connected by multiple connecting lines. The method may also include selecting colors for at least some of the points or at least some of the multiple connecting lines.

In one embodiment, the blood vessel may be an abdominal aorta, the procedure may involve applying an endograft to an abdominal aortic aneurysm, and the catheter may be an endograft delivery catheter. In some embodiments, selecting the first location may involve selecting a location on one side of an ostium of a blood vessel, and selecting the second location may involve selecting a location on an approximately opposite side of the ostium. In some embodiments, selecting the first and second locations may involve touching a touch screen. The method may also involve drawing a connecting line between at least two points on at least one of the images by drawing a line along the touchscreen.

These and other aspects and embodiments are described in greater detail below, in reference to the attached drawing figures.

### BRIEF DESCRIPTION OF THE DRAWINGS

FIG. 1A is an endoscopic image of an endovascular repair of an abdominal aortic aneurysm using an endograft;

FIGS. 1B and 1C are drawings representing fluoroscopic images of the endovascular repair of an abdominal aortic aneurysm of FIG. 1A;

FIGS. 2A and 2B are drawings representing fluoroscopic images of an endovascular repair of an abdominal aortic aneurysm using an endograft, illustrating a method of marking an image, according to one embodiment;

FIGS. 2C and 2D are drawings representing the same fluoroscopic images as in FIGS. 2A and 2B, respectively, but with additional points added, according to one embodiment of a method of marking an image;

FIGS. 2E and 2F are drawings representing the same fluoroscopic images as in FIGS. 2C and 2D, respectively, but with 3D Marks added, according to one embodiment of a method of marking an image;

FIG. 3 is an image of a blood vessel with multiple branching vessels, illustrating another method of marking an image, according to one embodiment;

FIGS. 4A and 4B are drawings representing fluoroscopic images of blood vessels, illustrating various methods for marking images, according to various embodiments;

FIGS. 5A and 5B are anterior-to-posterior (AP) and lateral (LR) views, respectively, of a catheter and a blood vessel, illustrating one example of how the location of an object relative to anatomy may be deceiving in one view alone; and

FIGS. 6A and 6B are drawings representing fluoroscopic and generated images, respectively, of a blood vessel with various catheter devices inserted therein, illustrating a method for providing multiple views with 3D Marks, according to one embodiment.

### DETAILED DESCRIPTION

Illustrative examples are shown in detail in the drawings below. Although the drawings represent the specific exemplary illustrations disclosed herein, the drawings are not necessarily to scale, and certain features may be exaggerated to better illustrate and explain an innovative aspect of an example. Further, the examples described herein are not intended to be exhaustive or otherwise limiting or restricting to the precise form and configuration shown in the drawings and disclosed in the following detailed description.

The description below and associated drawings generally describe ways of marking points or features in three-dimensional space to aid navigation of an elongated member, e.g., a catheter, without requiring a three-dimensional model.

In one exemplary illustration, a user interface and mathematical calculations are described that allow the physician to specify target anatomy in more than one two-dimensional view, e.g., using fluoroscopy. For example, the user may identify a point on fluoroscopy in two views and create a corresponding point (a “3D Mark”) in three-dimensional space. Multiple points could be created and linked together, to allow the user to mark exactly what is relevant to the case while avoiding the need to import a computed tomography (CT) image or use a significant amount of radiation or contrast for a rotational angiography. During a procedure, any 3D Marks could be viewed in conjunction with the sensed catheter location to provide alternate views of the task, without the need to move an associated catheter support or movement mechanism, e.g., a “C-arm.” The user may thus avoid the common situation of missing a target because the catheter or target location is out of plane with a two-dimensional imaging system, e.g., fluoroscopy. This approach can provide immediate benefit for ease of visualization with electromagnetic (EM) or other tip sensing enabled catheters and has the potential to reduce radiation and contrast exposure, such as in systems employing fluoroscopy.

In another exemplary illustration, a user interface may generally allow a user to easily identify three-dimensional features in the shared space of the imaging system (typically fluoroscopy) and the catheter based localization signal. Points can be identified by clicking or drawing lines on fluoroscopic views, marking the current location or shape of the catheter, or placing objects in 3D space. In each of these cases, the user may be provided with an easy-to-use user interface, which allows the user to essentially “draw” in 3D space. Then, alternate views can be shown, possibly overlaid on previously captured fluoroscopy images, to provide information to the user about the catheter shape and position outside of the two-dimensional plane, e.g., a fluoroscopy plane.

One advantage of this approach is that it does not require importing three-dimensional data from an outside source. Doctors are generally hesitant to capture 3D data using rotational angiography during the procedure, because it increases the dosage of radiation and contrast that the patient receives. Pre-operative CTs, while often available, need to be registered to the fluoroscopy coordinate frame and may contain stale data, because they were captured days or weeks earlier. Furthermore, that data also needs to be segmented, which can be a labor intensive and technically demanding job. Finally, importing the data from outside sources requires significant implementation within a given system produced by a manufacturer, and generally requires extensive third party business development.

While examples may be employed in catheter systems where only two-dimensional imaging is available, it is worth noting that one of the common workflows for use of three-dimensional imaging systems is to mark specific areas of interest on the three-dimensional model, and then show only those markings overlaid on a two-dimensional image, e.g., fluoroscopy (see, for example, Siemens' DynaCT and syngo technology). Accordingly, the exemplary approaches described herein would provide a convenient user interface with much of the same functionality, even though a three-dimensional imaging system is available. In this manner, usage of the three-dimensional imaging system may be used minimally, resulting in reduced energy consumption and/or reduced radiation for the physician and patient and reduced contrast used on the patient.

Further exemplary illustrations and background are provided in the description below. The exemplary illustrations described herein are not limited to the examples specifically described. Rather, a plurality of variants and modifications are possible, which also make use of the ideas of the exemplary illustrations and therefore fall within the protective scope. Accordingly, the description is intended to be illustrative and not restrictive.

With regard to the processes, systems, methods, heuristics, etc. described herein, although the steps of such processes, etc. may be described as occurring according to a certain ordered sequence, such processes could be practiced with the described steps performed in an order other than the order described herein. Furthermore, certain steps could be performed simultaneously, other steps could be added, or certain steps described herein could be omitted. In other words, the descriptions of processes herein are provided for the purpose of illustrating certain embodiments, and should in no way be construed as limiting the claimed invention.

Accordingly, the description herein is intended to be illustrative and not restrictive. Many embodiments and applications other than the examples provided would be upon reading the description herein. The scope of the invention should be determined, not with reference to the above description, but should instead be determined with reference to the claims, along with the full scope of equivalents to which such claims are entitled. It is anticipated and intended that future developments will occur in the arts discussed herein, and that the disclosed systems and methods will be incorporated into such future embodiments. In sum, the invention is capable of modification and variation and is limited only by the claims.

All terms used in the claims are intended to be given their broadest reasonable constructions and their ordinary meanings as understood by those skilled in the art, unless an explicit indication to the contrary in made herein. In particular, use of the singular articles such as “a,” “the,” “the,” etc. should be read to recite one or more of the indicated elements unless a claim recites an explicit limitation to the contrary.

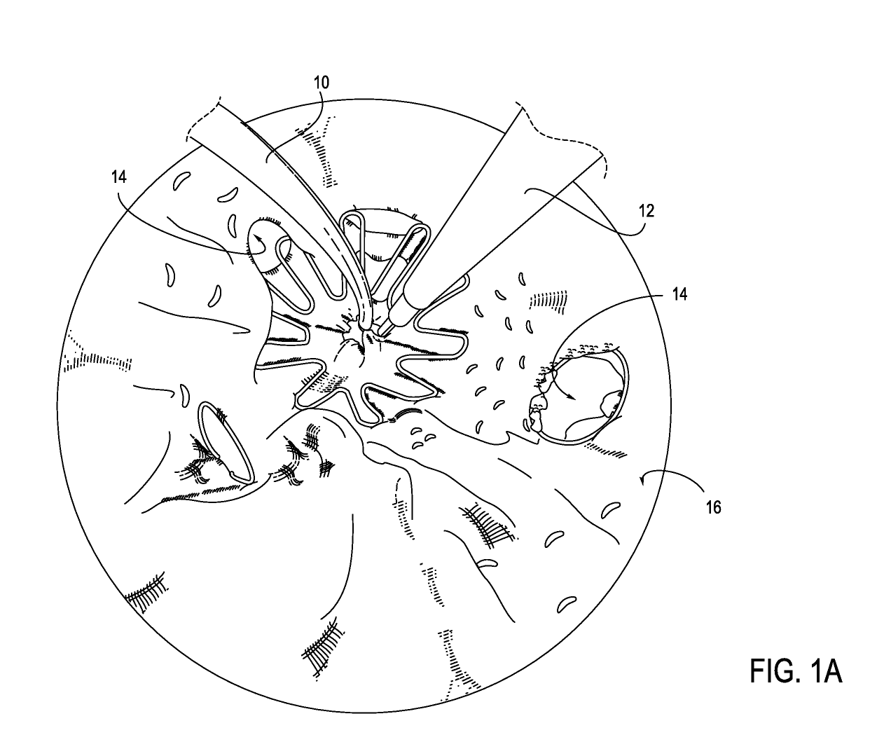

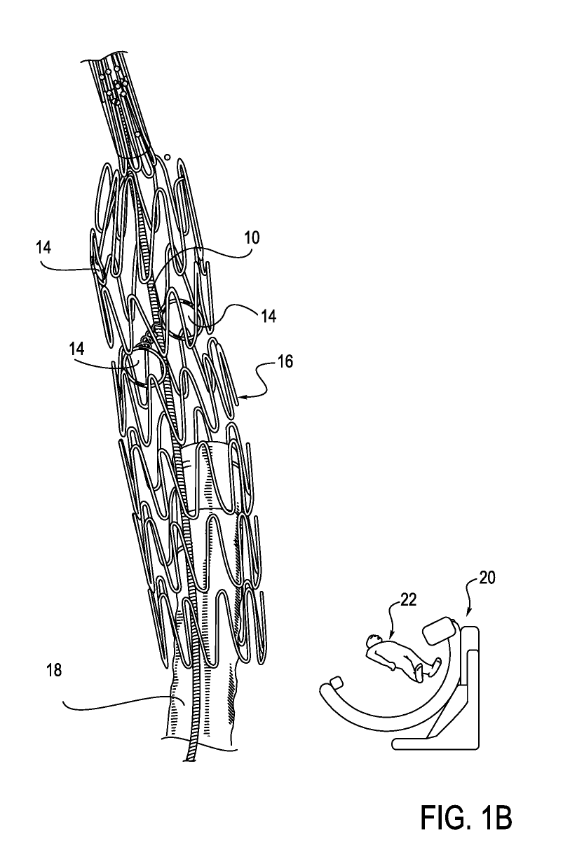

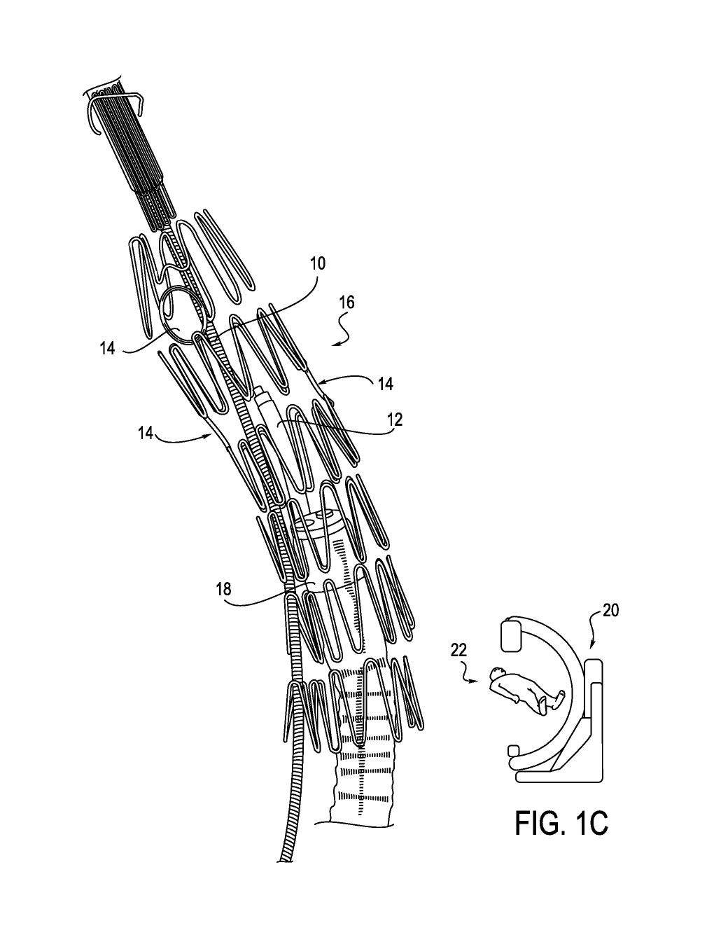

Referring now to FIG. 1A, an endovascular view of an abdominal aortic aneurysm (AAA) repair using an endograft is shown. While physicians typically perform such a procedure using only a 2-D fluoroscopic image, many tasks in endovascular procedures, such as the illustrated AAA repair, require motion in all three dimensions. FIG. 1A shows several features that need to be navigated around in three dimensions. For example, FIG. 1A shows a graft delivery catheter 10, a procedural catheter 12, fenestrations 14 and an endograft 16. On fluoroscopy, most of the 3-D structure is lost, and even if the physician rotates the C-arm, it is difficult to understand and remember the three-dimensional relationship of the elements of the procedure. For instance, in the fluoroscopic projection, it is difficult to determine if one catheter 10 is in front of or behind another element, such as a fenestration 14 or other catheter 12. Yet, if the user does not realize the 3D relationship, there may be no way to navigate the catheter 10 from the current position to achieve his or her goal.

FIGS. 1B and 1C are drawings representing fluoroscopic views of the same physical structure as in the endoscopic view of FIG. 1A. In addition to the features described above, FIGS. 1B and 1C show an endoscope 18. Fluoroscopic images, such as those illustrated by FIGS. 1B and 1C, may be acquired using a standard fluoroscopic imaging system, such as a C-arm 20, which are well known to those skilled in the art. A small cut-out illustration of a C-arm fluoroscopic system 20 and its position relative to a patient 22 is included in each of FIGS. 1B and 1C, to show the orientation of each image.

When the coordinate frame for a localization system is registered to the coordinate frame of an imaging system such as fluoroscopy, a common three-dimensional space is created between sensing and imaging coordinate frames. One of the valuable traits of localization is that it allows interaction between the catheter and image features within this three-dimensional space. One way to do this is to populate this common space with a three-dimensional model of the anatomy, such as a pre-operative CT or rotational angiography volume.

This application describes alternate methods of populating this three-dimensional space with three-dimensional geometric constructions that can be aligned with relevant anatomical features. This can provide much of the benefit of three-dimensional models while avoiding the increased radiation and contrast needed to generate them. In addition, the simple nature of such geometric constructions makes them easier to use. A number of geometric primitives and methods to define them are described.

One element that is important to the success of this approach is reliable calculation of the three-dimensional shape, location, and orientation of the constructed objects, or “3D Marks”, while providing a user interface that is easy to use. Since the primary users would be physicians or hospital staff, simplicity of defining and adjusting the three-dimensional shape and position of the 3D Marks may be more important than complex features.

This description is written with a few assumptions about localization technology and imaging technology. There are other approaches, such as other types of localization technology or imaging technology or alternate interfaces, but the core features would resemble what is described here. It is assumed that the localization technology described is electromagnetic sensing that provides, in one example, at least five millimeters (mm) of absolute accuracy (“trueness”), but other localization technologies are relevant as well. Five mm of error in position is quite large, and certain applications related to precise positioning may require more accuracy. In certain areas of the anatomy that move significantly during the breath cycle or heartbeat, a compensation algorithm may be needed to adjust for motion of the anatomical structures.

Some of the approaches described may generally require the ability for the user to designate certain locations on two-dimensional images such as fluoroscopy. Generally, the interface will be described as “clicking” or “drawing a line” on the images, although any number of pointing methods could be used, such as but not limited to: a mouse; a trackball, e.g., as used in the Magellan™ pendant or Sensei™ pendant, commercially available from Hansen Medical, Inc.; a touchpad, e.g., similar to that on a laptop; a touchscreen on either the main screen or an auxiliary screen that can detect touches or gestures on the image itself; three-dimensional or stereo detection technologies; a handheld pointing device; and/or haptic input devices.

Because the imaging available is two-dimensional, most of these devices would be used to designate two-dimensional points on multiple images taken at different viewing angles. In some cases, the devices that allow three-dimensional input, such as a haptic input device, could designate a three-dimensional point directly, using one or more views on the screen or biplane fluoroscopy. It may be challenging, however, to identify three-dimensional points with a two-dimensional view on the screen. One alternative embodiment may use a three-dimensional display, such as a stereo display or holographic display.

While many of the imaging modalities can apply, this discussion will focus on two-dimensional imaging, and in particular on fluoroscopy, because it is the most widely used imaging modality in vascular procedures. However, any number of other modalities may also work with this approach, including, without limitation, IVUS (intravenous ultrasound), OCT (optical coherence tomography), or any other imaging modality that can be registered to a common frame of the sensor. It is also assumed that there are at least two viewing areas on one or more screens accessible to the user that can render fluoroscopy or 3D views. Multiple viewing areas allow the user to see more than one view of the three-dimensional space simultaneously. In yet another exemplary illustration, a three-dimensional display using stereo vision or a holographic display could provide the three-dimensional information.

In one exemplary approach, a set of 3D Marks can be created by providing the user with an interface to identify anatomy on fluoroscopic images and designate it with a mouse or other pointing device in multiple views. Identifying the same feature in multiple views allows one of a number of mathematical computations to be used to determine the 3D location of that item. In some embodiments, the interface provides an easy-to-use method for capturing a single frame of the imaging in each view and then allowing the user to mark on the stored images side by side. This significantly reduces radiation exposure as well. Showing the images side by side makes it easy to compare the views and identify anatomy more readily. Storing images may be necessary, e.g., because the user may often mark on images from contrast injection, and it would be undesirable to inject contrast multiple times. This could be completed at an exemplary work station, e.g., a Magellan™ WorkStation, by showing the two images next to each other in the designated spaces. The configuration of the fluoroscopy system is stored for each image to allow registration of points between the images.

In some embodiments, it may be possible to do some, or all, of the feature matching through computer vision technologies. Initially, though, the easiest implementation may be to use the physician as a vision recognition system and reduce the risk of an inaccurate identification. Furthermore, the physician will have the opportunity to mark exactly what features they are interested in and not mark those that are not relevant to his or her current task.

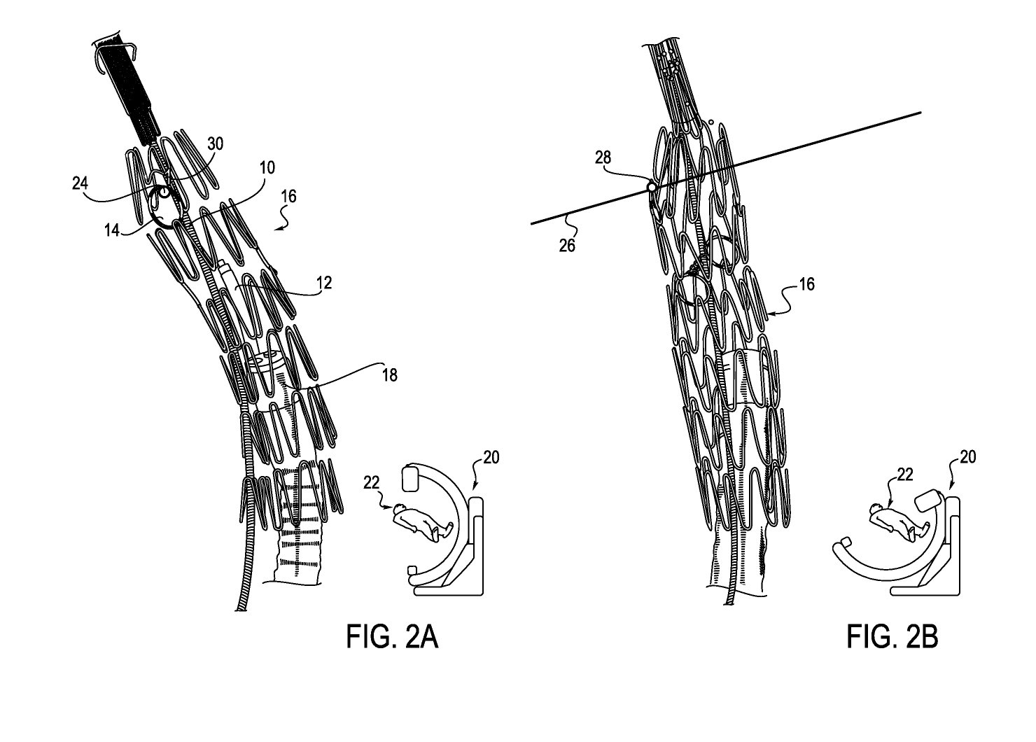

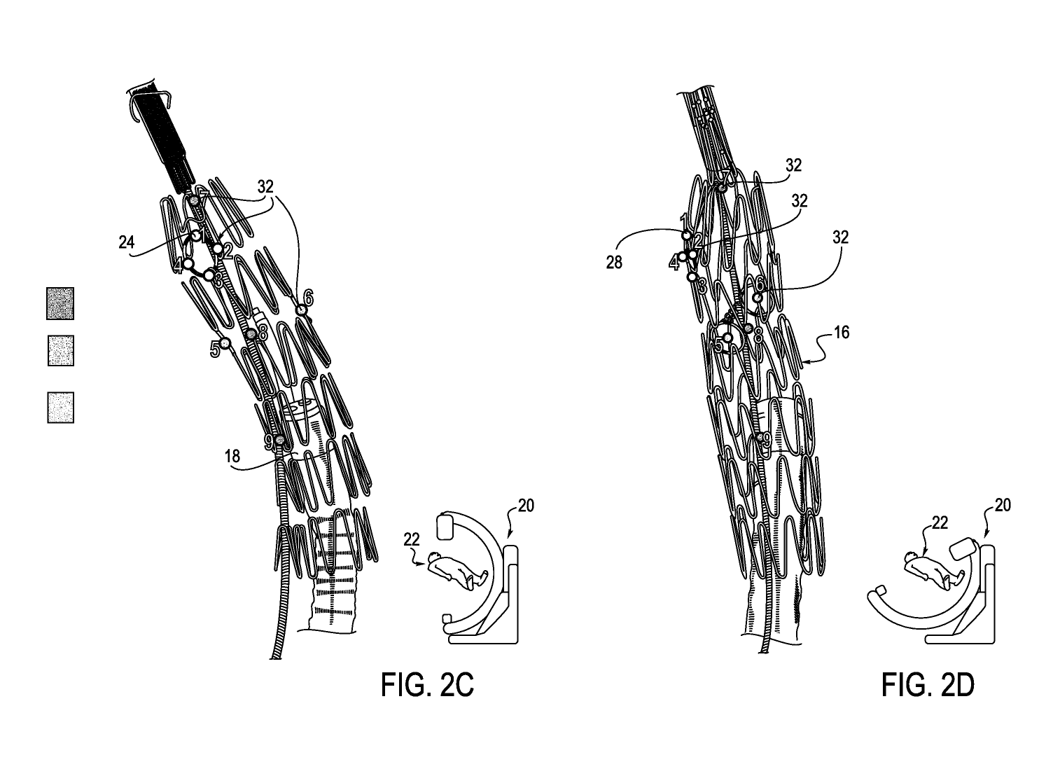

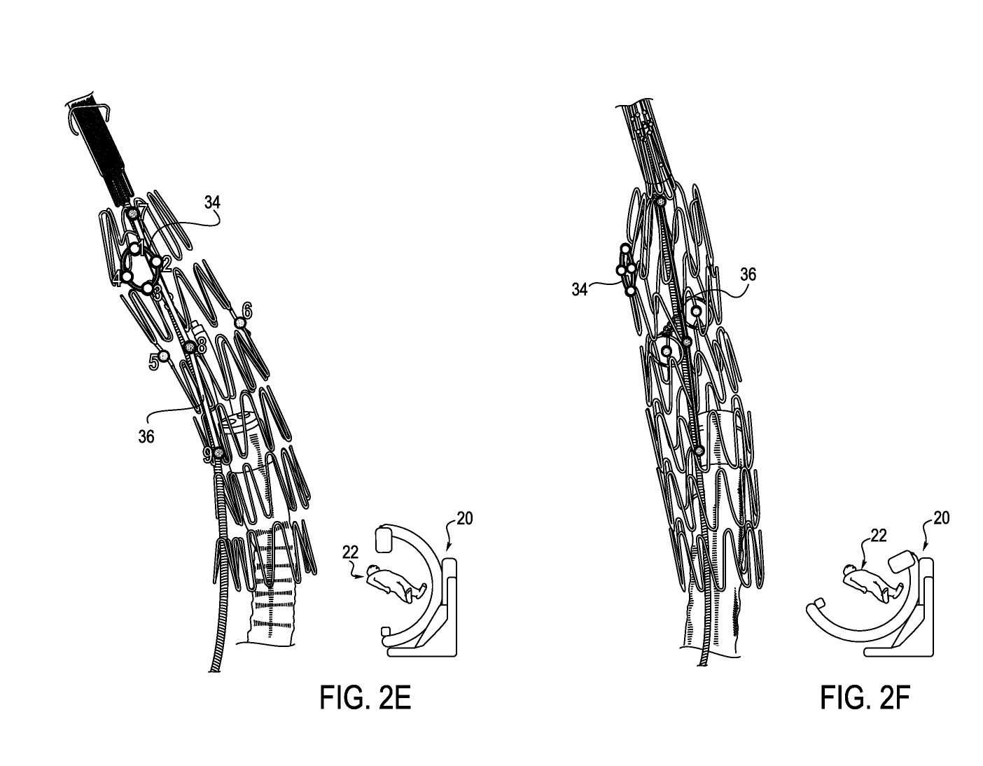

Referring now to FIGS. 2A and 2B, one embodiment of a method for creating 3D Marks of different types involves the user designating points in three-dimensional space and then linking them together to create shapes. FIGS. 2A and 2B are examples of two fluoroscopic images, which in a typical embodiment would be shown next to one another on the same display screen or different screens, for viewing by a user. FIGS. 2A and 2B illustrate one embodiment of a process for designating a single point.

As a first step, on the image in FIG. 2A, the user may click on a designated feature in one of the images to generate a point 24 (or “dot”) on the image. A line of projection 26 (or “guide line”) is then generated on the image in FIG. 2B. The guide line 26 is a line that the corresponding point 24 should be on. Using the guide line 26 as a guide to identify the corresponding feature, the user can click on the feature on the image of FIG. 2B, thus generating a second point 28. The system computes a three-dimensional location that is closest to the two designated points 24, 28. One way to do this is to project both points 24, 28 to projected lines in 3-D space based on the fluoroscopy projection, then find the point that is a minimum distance from both lines. This 3D point (“3D Mark”) is then assigned a unique number 30, for example “1,” as illustrated in FIG. 2A. The user may repeat this process for as many points as needed.

An alternative way of designating points, which would not require placing two images side-by-side, would be to click a number of points in the image of FIG. 2A and then sequence through them (with the guide lines) to identify them in the image of FIG. 2B. The guide lines 26 help identify features that are related between the two images and they also give the user another opportunity to identify a feature, in case they identified the wrong feature in the previous image. For added accuracy, the same process could identify points in more than two images and then find the best fit.

Referring now to FIGS. 2C and 2D, in some embodiments, the user can determine how points identify the anatomical features and label them or identify them by color if needed. (Different colors are represented herein as different shadings in FIGS. 2C and 2D and other figures.) Specifying colors and/or labels that are consistent across all views may help a user identify important features. FIGS. 2C and 2D show more items identified with additional points 32. (In this example, points 32 include points labeled 2-9 in the drawing, although for clarity the label “32” does not point to all of points 2-9). In this example, points 1-4 are around a fenestration, points 5-6 identify the centers of specific fenestrations, and points 7-9 identify the endograft catheter shaft.

Referring now to FIGS. 2E and 2F, points (which can be represented as spheres in three-dimensional space) may provide good identification of features, but it is also useful to be able to link them together to make more complex constructions. For example, in some embodiments, lines may link multiple points in sequential fashion. In one example, this kind of linking may define a heading of a Transcatheter Aortic Valve Implantation (TAVI) valve. In various embodiments, three or more points together can define a spline and/or three or more points together can define a circle or ellipse. In the example show in FIGS. 2E and 2F, two “3D Marks” are shown. An ellipse 34 is drawn through points 1-4 to designate a fenestration, and an approximately straight line 36 is drawn through points 7-9 to designate the graft delivery catheter shaft.

While linking points to create more complex 3D Marks is probably the most straightforward approach to identify a feature, there are other options for defining shapes in three dimensions, based on user interaction. For example, the user could draw lines or curves along features of interest in both images to create a three-dimensional line or curve. Typically, there would not be one unique solution to the three-dimensional shape that is defined by two, two-dimensional curves, but a least squares technique would find a solution that best fits the input. This approach would be useful to draw lines down a fixed catheter or other elongate members of interest that do not move, such as the shaft of the graft catheter during endograft deployment. A similar process could be used to identify the gates in an endograft by drawing a circle or ellipse in each view and then solving for the best fit in three dimensions using a least squares technique. In alternative embodiments, other computational geometry algorithms for matching curves and shapes to construct a three-dimensional shape from two or more two-dimensional shapes may be applied.

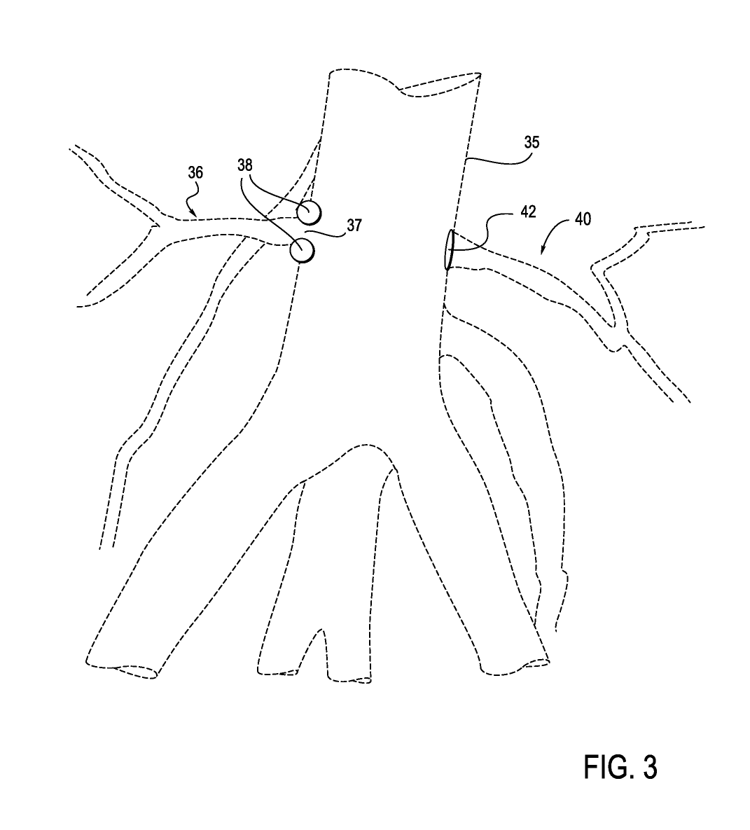

Referring now to FIG. 3, another approach could be focused on identifying ostia of blood vessels (or other structures in alternative embodiments) with circles or ellipses in three dimensions by using a process where the physician identifies the ostium in two views. FIG. 3 is a contrast-enhanced image of a primary blood vessel 35, with a first branching vessel 36 and a second branching vessel 40. Two examples of ways of marking ostia are that the physician could specify edges of an ostium 37 in two views by placing a point 38 at each edge of the ostium 37 (shown here for the first branching vessel 36) or a line 42 across an ostium (shown here for the second branching vessel 40). If an ostium is identified in two or more views, a least-squares technique could be used to solve for the best fit ellipse that includes the heading of the opening of the ostium. In some embodiments, more than two views may be used, for increased accuracy. Projecting the points or line in one view to a candidate area in another view may make it easier to identify the feature desired. Mathematical algorithms other than least squares, which are suitable for providing the best three dimensional shape that fits multiple 2-D points or shapes, may also be used in some embodiments.

Providing aids such as the projected possible locations of features (a point in one view would be a line in another view) can prevent the user from getting confused about which feature has been identified. Furthermore, showing these aids and updating them in real time as the fluoroscopy angle is changed would allow the user to determine the best alternate view that can be used to distinguish similar features. This would allow the user to choose the view in which the relevant features are best separated to avoid delays or inaccuracy in specifying the 3D Marks.

It is also possible to create 3D Marks using the electromagnetic sensors themselves. For instance, in a task where the user is trying to navigate to a specific vessel but there are many branches in the area (such as an embolization case), it would be useful to mark the vessels that are not relevant to the treatment or have already been treated. If an instrumented wire is used in the procedure, the user could pull back that wire to create a path in three-dimensional space (collecting a group of 3D Marks based on the position) once a vessel is treated. Then, in later navigation using other views, the user could easily determine if they went down that vessel again. The user could also use data collection to map the free space by moving around the catheter in a lumen.

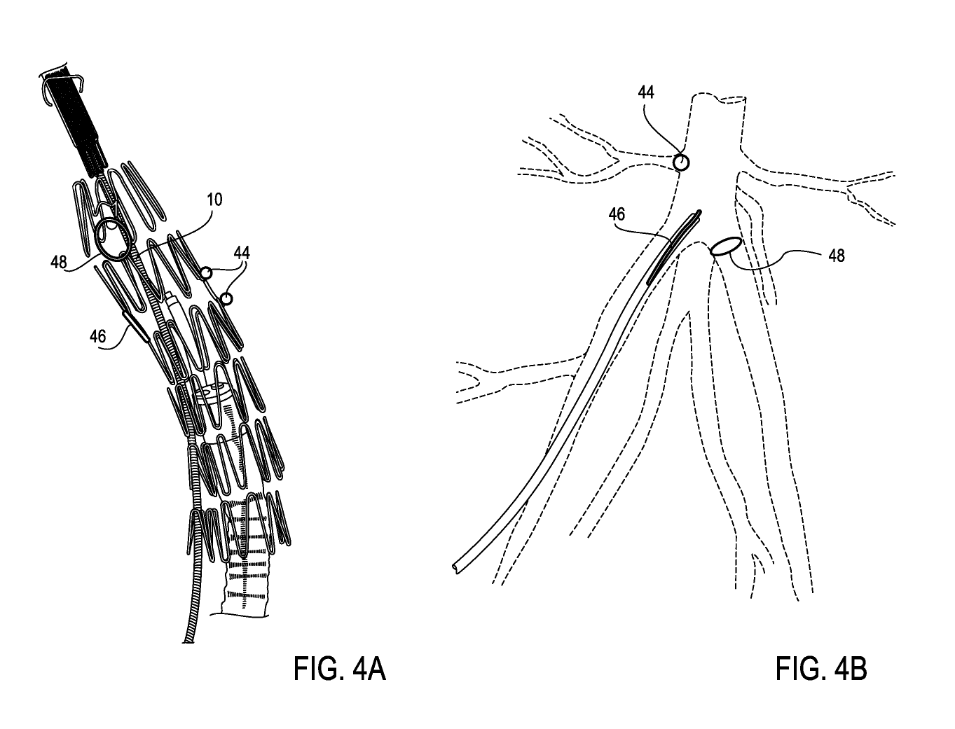

Referring now to FIGS. 4A and 4B, various embodiments of the methods described herein for designating and representing three-dimensional shapes and objects, or “3D Marks,” may involve any of a number of suitable methods for presenting information to the user. One method, for example, involves overlaying the 3D Marks on live fluoroscopy, to act as a guide when navigating instrument(s) in the body. In the examples shown in FIGS. 4A and 4B, for example, points 44, lines 46 and circles 48 are used to mark various features. In one embodiment, the points 44 may be colored green, the lines may be colored blue 46, and the circles 48 may be colored red. This is simply one exemplary embodiment, however, and in various alternative embodiments, any variety of shapes, colors, transparencies and/or animation may be used.

According to some embodiments, because the user defines what 3D Marks are displayed, the user can determine if they would like to mark anatomical features that are already visible in fluoroscopy, such as a fenestration in an endograft, or anatomical features, such as blood vessels, that are not visible on fluoroscopy without a contrast injection.

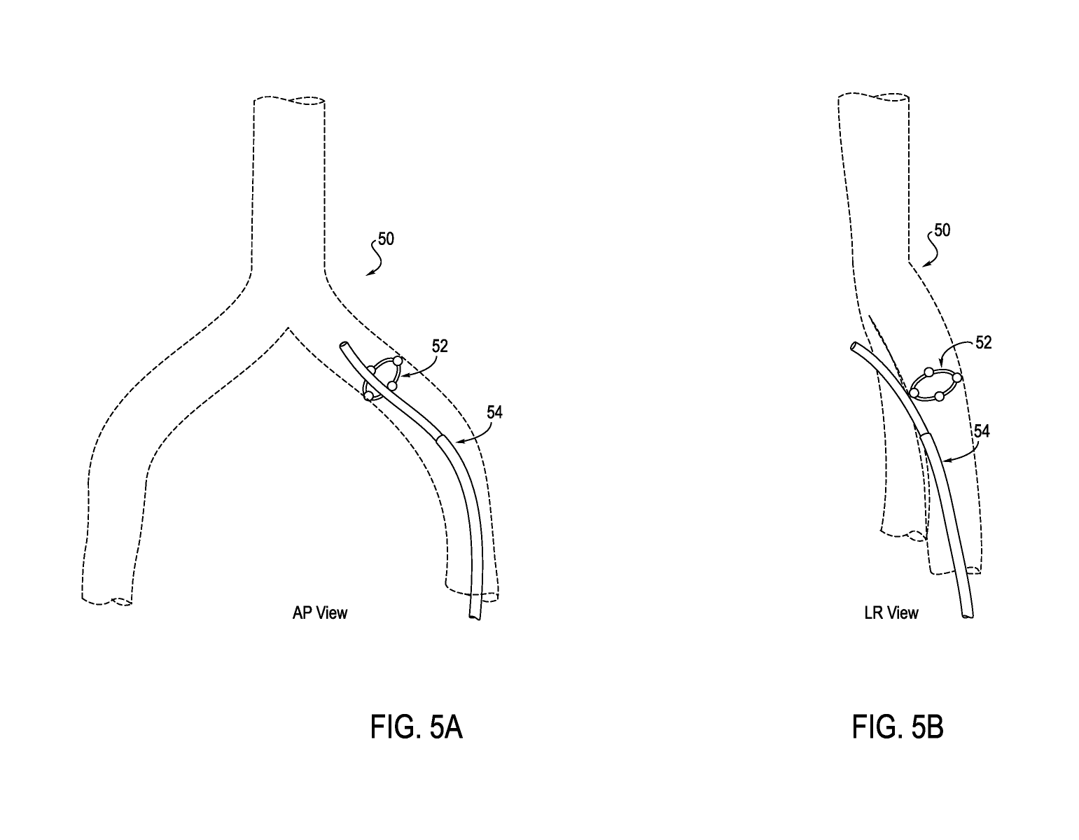

Referring now to FIGS. 5A and 5B, one important feature of the method described herein is the ability to provide one or more alternative views that are different than the imaging view that is being provided at that time by the fluoroscopy system. One of the biggest challenges in performing endovascular procedures using a fluoroscopic image is that it is not easy to see movement of the catheter when the catheter is moving in a direction out of the plane of the fluoroscopic image. For example, it is very common for a physician to struggle with a contralateral gate in an AP (anterior-to-posterior) view and then switch to an oblique view and find that the catheter is significantly anterior or posterior to the gate. FIGS. 5A and 5B depict this situation. FIG. 5A depicts an AP view of a blood vessel 50 with a contralateral gate 52. A catheter 54 appears to be extending through the gate 52 from this view. As shown in FIG. 5B, however, which is a lateral (LR) view, the catheter 54 is actually anterior to the gate 52.

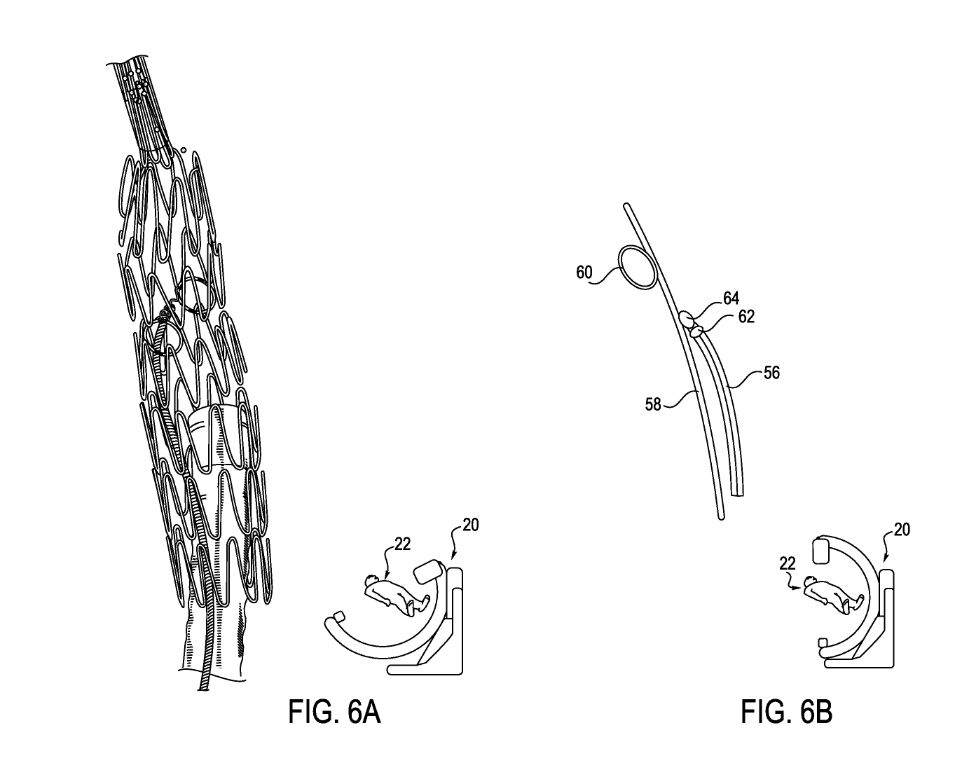

Referring now to FIGS. 6A and 6B, presenting an alternate view, using the 3D Marks and the localized catheter position, allows the user to see the relationship between the catheter and the target without changing the imaging view or using additional radiation or contrast. The precision of these alternate views does not need to be very high, because the primary view is a fluoroscopic image, which provides the needed precision. The alternate views do, however, provide information to help the user manipulate the catheter out of plane with the fluoroscopic image. One embodiment of a two-view presentation is illustrated in FIGS. 6A and 6B. FIG. 6A is a fluoroscopic image taken from a lateral right orientation, and FIG. 6B is an alternate, non-fluoroscopic image provided in an anterior-to-posterior orientation. In the alternate view of FIG. 6B, various elements are marked—e.g., a catheter 56, a catheter leader tip 64, a sheath tip 62, a target fenestration 60, and a deployment catheter 58 for the endograft. In various embodiments, these and/or other elements may be marked and given various colors. In one embodiment, for example the catheter 56 may be blue, the deployment catheter 58 may be yellow, the fenestration 60 may be purple, the sheath tip 62 may be red, and the leader tip 64 may be green.

As with many of the previous figures, each of the images of FIGS. 6A and 6B includes a diagram of a C-arm 20 and a patient 22, to illustrate the orientation of the different views. Some embodiments may additionally or alternatively show a virtual plane or projected rectangle on either view to show one view in relation to the other. A third way of depicting the difference is to use an orientation indicator, such as the cube with letters on it that many companies use to depict fluoroscopy viewing directions. For clarity, it could also use the same markings either overlaid on the primary view (fluoroscopy) or a third view that has the same orientation as the primary view. In fact, any number of alternate views could be generated, depending on the user's needs and/or preferences. This would make it easy for a user to understand that these are two different views of the same thing. Yet, because a secondary view is completely virtual, it could be independently rotated and adjusted to provide an ideal view of what the user is working with. Or, this approach could be used to make a “virtual biplane” with a fixed 90° difference between the two views, even as the fluoroscopy angle changes. Instinctive driving (e.g., motions of the user mapping to the equivalent motion in imaging—i.e. moving the input to the left would cause the catheter to move left in the imaging) would typically occur in relation to the primary view, not the alternate view.

In some embodiments, the background of the alternate view is empty. Alternatively, however, it may also be possible to use one or more fluoroscopic images or other images to generate an alternate view with a virtual background. In some embodiments, the imaging could be faded, colored, or otherwise marked to signify that it is not live imaging, while at the same time providing context to the virtual alternate view showing the 3D Marks. In some embodiments, previous fluoroscopic images could be looped in the background of the alternate view and synchronized based on breathing motion or heartbeat with an external sensor (such as an impedance sensor or second EM sensor attached to the chest).

In order to make the user interface easy to use, it is important to put the ability to view three-dimensional information in the hands of the physicians where they need that information. For that reason, it is important to support displaying these alternative views at bedside and at the work station. In both locations, a pointing device and some method of reorienting any alternative 3D views should be provided. It is also possible for two physicians to work collaboratively; one at bedside, sterile, configuring catheters and providing treatment, and a second at the workstation, unsterile, marking on images for 3D Marks. It also allows some physicians to become more familiar with the imaging and marking procedure to allow the facility to increase throughput through specialization. Either physician could navigate the catheter in this situation, but significant procedure time could be saved by allowing two physicians to work independently.

Given that the data is three-dimensional, 3D viewing technologies, such as but not limited to stereoscopic glasses, holograms, and/or 3D displays could be used to display more information. In some embodiments, the user may also be provided with a method to manually adjust the locations of the 3D Marks for simple situations, such as the patient shifting on the table. For example, by placing 3D Marks on bony landmarks the doctor could provide alignment without significant additional radiation.

1. A method for facilitating a medical or surgical procedure in an operating site in a body of a patient, the method comprising:
displaying a first point on a first two-dimensional image of the operating site into which an elongate, flexible catheter device is inserted in response to a first user input;
mapping the first point on at least a second two-dimensional image of the operating site, the second two-dimensional image being oriented at a non-zero angle with respect to the first two-dimensional image;
displaying a first line on the second image that projects from the first point;
displaying a second point on the second image in response to a second user input; and
determining a three-dimensional location within the operating site, based on the first line and the second point on the second image.

2. The method of claim 1, wherein the first two-dimensional image comprises an image generated with a fluoroscopic imaging system.

3. The method of claim 1, wherein the catheter device is selected from the group consisting of a procedural catheter, an endograft delivery catheter, a catheter sheath and a catheter guidewire.

4. The method of claim 1, wherein the catheter device comprises an electromagnetic sensor.

5. The method of claim 1, further comprising displaying a connecting line connecting the first and second points on the second image in response to an additional user input.

6. The method of claim 5, further comprising displaying a number label for one or more of the points on at least one of the first image or the second image.

7. The method of claim 6, further comprising displaying all the points and all the number labels on both the first image and also the second image.

8. The method of claim 1, wherein displaying the first and second points comprises displaying the points with different colors.

9. The method of claim 1, further comprising displaying the first and second images, wherein the first and second images are displayed on one display monitor.

10. The method of claim 1, further comprising displaying the first and second images, wherein the first and second images are displayed on two display monitors.

11. The method of claim 1, wherein the first image is derived from an imaging modality, and wherein the method further comprises:
generating the second image from the first image; and
displaying the second image.

12. The method of claim 11, wherein generating and displaying the second image comprises generating and displaying a representation of at least part of the catheter device and at least part of the operating site.

13. The method of claim 12, wherein generating and displaying the second image further comprises generating and displaying a representation of a background of the operating site.

14. The method of claim 1, wherein the operating site comprises an abdominal aortic aneurysm, and wherein the catheter comprises an endograft delivery catheter.

15. The method of claim 1, wherein the first image comprises an image acquired from an imaging system oriented in a first orientation relative to the patient selected from the group consisting of anterior-posterior and posterior-anterior, and wherein the second image comprises an image oriented in a second orientation relative to the patient selected from the group consisting of left lateral and right lateral.

16. The method of claim 1, wherein displaying the points comprises displaying points at an ostium of a blood vessel.

17. The method of claim 1, further comprising displaying an ellipse that connects the first point and the second point on at least one of the first image or the second image.

18. The method of claim 1, wherein determining the three-dimensional location comprises using a least squares method of mathematical calculation.

19. A system for facilitating a medical or surgical procedure in an operating site in a body of a patient, the method comprising:
an elongate, flexible catheter device; and
a visualization system, comprising:
a user interface for accepting user inputs related to locations of items on at least two displayed images;
an image generator configured to generate images of at least a first point on a first image of the operating site and a second point on a second image of the operating site in response to the user inputs, wherein the first image is acquired via an imaging modality, and wherein the second image has an orientation that is different from an orientation of the first image; and
a processor configured to map the first point on the first image with an equivalent first point on the second image, generate a first line for display on the second image that projects from the first point, and determine a three-dimensional location within the operating site, based on the first line and the second point on the second image.

20. The system of claim 19, wherein the catheter device is selected from the group consisting of a procedural catheter, an endograft delivery catheter, a catheter sheath and a catheter guidewire.

21. The method of claim 19, wherein the catheter device comprises an electromagnetic sensor.

22. The system of claim 19, wherein the visualization system further comprises at least one video display monitor.

23. The system of claim 22, wherein the visualization system comprises:
a first video display monitor for displaying the first image; and
at least a second video display monitor for displaying the second image.

24. The system of claim 19, wherein the imaging modality comprises fluoroscopy, and wherein the visualization system comprises a connection for connecting to a fluoroscopic imaging system.

25. The system of claim 19, wherein the processor of the visualization system is further configured to generate at least a second line connecting multiple points on at least the second image in response to the user inputs.

26. The system of claim 19, wherein the user interface comprises at least one device selected from the group consisting of a touchscreen, a keyboard and a mouse.
